(19) United States
(12) Patent Application Publication
(10) Pub. No.: US 2026/0076935 A1
(43) Pub. Date: Mar. 19, 2026

(54) PREVENTION AND TREATMENT OF CONDITIONS USING ETHYL PYRUVATE

(71) Applicant: The Trustees of Columbia University in the City of New York, New York, NY (US)

(72) Inventors: Simon W.M. JOHN, Bar Harbor, ME (US); Nicholas TOLMAN, New York, NY (US)

(21) Appl. No.: 19/334,248

(22) Filed: Sep. 19, 2025

Related U.S. Application Data
(63) Continuation of application No. PCT/US2024/020798, filed on Mar. 20, 2024.
(60) Provisional application No. 63/453,419, filed on Mar. 20, 2023.

(51) Int. Cl. A61K 31/22; A61K 31/198; A61K 31/455; A61K 31/4745; A61K 31/706; A61K 31/7084; A61K 45/06; A61P 27/06

(52) U.S. Cl. CPC A61K 31/22; A61K 31/198; A61K 31/455; A61K 31/4745; A61K 31/706; A61K 31/7084; A61K 45/06; A61P 27/06

(57) ABSTRACT

Methods of treating or preventing conditions in a subject are disclosed, the methods including administering to the subject a therapeutically effective amount of ethyl pyruvate, or a derivative or analog thereof, or a pharmaceutical composition thereof. Methods of treating or preventing conditions in a subject including administering to the subject a therapeutically effective amount of ethyl pyruvate, or a derivative or analog thereof, or a pharmaceutical composition thereof, and one or more additional therapeutic agents, are described.

### CROSS REFERENCE TO RELATED APPLICATION

The application is a continuation of International Patent Application No. PCT/US2024/020798, filed on Mar. 20, 2024, which claims the benefit of and priority to U.S. Provisional Application No. 63/453,419, filed on Mar. 20, 2023, the content of which is hereby incorporated by reference its entirety.

### STATEMENT REGARDING FEDERALLY SPONSORED RESEARCH OR DEVELOPMENT

This invention was made with government support under EY032062 and EY032507 awarded by the National Institutes of Health. The government has certain rights in the invention.

### INCORPORATION BY REFERENCE

Any patent, patent publication, journal publication, or other document cited herein is expressly incorporated herein by reference in its entirety.

### FIELD

This present application is generally related to the prevention and treatment of conditions using ethyl pyruvate and combinations of ethyl pyruvate with one or more additional therapeutic agents.

### BACKGROUND

Metabolic intermediates, such as sodium pyruvate, calcium pyruvate, and nicotinamide have therapeutic potential for certain diseases or conditions. For example, these compounds have been effective at treating glaucoma in certain preclinical animal models, by reducing intraocular pressure (“IOP”) through metabolic modulation and/or providing neuroprotection. See, e.g., WO 2017/070647 A1; Jeffrey M. Harder et al., Disturbed Glucose and Pyruvate Metabolism in Glaucoma with Neuroprotection by Pyruvate or Rapamycin, 117 Proc. Nat'l Acad. Sci. 33619 (2020); Pete A. Williams et al., Vitamin B3 Modulates Mitochondrial Vulnerability and Prevents Glaucoma in Aged Mice, 355 Science 756 (2017).

However, there are no direct neuroprotective treatments approved for glaucoma. Additionally, new treatments would be beneficial to prevent damage to the tissues involved in ocular fluid drainage and/or to reduce IOP. Current treatments outcomes are often patient-specific and short-lived, necessitating surgical interventions, which themselves may fail over time. There are no current treatments to prevent developmental malformations that have certain functional, cosmetic, and/or psychological impacts. Moreover, molecules like sodium pyruvate suffer from sub-optimal physiological properties and efficacy. Therefore, improved therapies and therapeutic strategies are desirable that not only address the aforementioned ocular conditions, but that enable use of metabolic intermediates for other types of diseases or conditions.

### SUMMARY

In one aspect, a method of treating or preventing a condition in a subject is described, the method comprising administering to the subject a therapeutically effective amount of ethyl pyruvate, or a derivative or analog thereof, or a pharmaceutical composition thereof; wherein the condition is selected from the group consisting of an ocular disease or disorder, a neurodegenerative disease or disorder, a vascular disease or disorder, a metabolic disease or disorder, an inflammatory disease or disorder, a disease or disorder involving abnormal cell death or oxidation, a disease or disorder linked to one or more Lmx1b mutations, and a combination thereof.

In another aspect, a method of reducing or preventing elevated intraocular pressure is described, the method comprising administering a therapeutically effective amount of ethyl pyruvate, or a derivative or analog thereof, or a pharmaceutical composition thereof, to the subject.

In another aspect, a method of reducing or preventing damage or dysfunction to a subject's eye tissues is described, the method comprising administering a therapeutically effective amount of ethyl pyruvate, or a derivative or analog thereof, or a pharmaceutical composition thereof, to the subject. In some cases, the subject is pregnant and the ethyl pyruvate is administered during prenatal care to protect the child from developing dysfunction/disease. Treatment can continue for the child after birth either by direct administration to the child or through the mother via breast milk or a combination thereof.

In another aspect, a method of reducing or preventing damage or dysfunction to tissues involved in ocular fluid drainage in a subject is described, the method comprising administering a therapeutically effective amount of ethyl pyruvate, or a derivative or analog thereof, or a pharmaceutical composition thereof, to the subject.

In another aspect, a method of reducing or preventing developmental malformations, disease dependent malformations or dysfunctions in a subject's eye is described, the method comprising administering a therapeutically effective amount of ethyl pyruvate, or a derivative or analog thereof, or a pharmaceutical composition thereof, to the subject.

In any one of the embodiments described herein, the method further comprises administering one or more additional therapeutic agents, or one or more pharmaceutical compositions thereof, to the subject.

In any one of the embodiments described herein, the one or more additional therapeutic agents are selected from the group consisting of an antioxidant agent, an anti-inflammatory agent, an agent that modulates metabolism, an agent that modulates the integrated stress response, an agent that modulates the unfolded protein response, an agent that modulates forms of autophagy, an agent that modulates the expression or activity of genes controlling or mediating antioxidant or other protective responses, an anti-cell death agent, a senolytic agent, an agent that modulates the mitochondria or mitophagy, an anti-aging agent, an agent that modulates intraocular pressure, a resilience-boosting agent, an antifibrotic agent, an agent that prevents epithelial mesenchymal transition or endothelial mesenchymal transition, a neuroprotective agent, a gene therapy agent, and a combination thereof.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DESCRIPTION OF THE DRAWINGS

The application is described with reference to the following figures, which are presented for the purpose of illustration only and are not intended to be limiting. In the Drawings:

FIG. 1 shows ethyl pyruvate protected against high IOP, according to one or more embodiments.

FIG. 2 shows ethyl pyruvate alone or in combination with nicotinamide lessened IOP elevation, according to one or more embodiments.

FIG. 3 shows ethyl pyruvate in combination with other therapeutic agents afforded increased disease protection, according to one or more embodiments.

FIG. 4 shows ethyl pyruvate rescued ocular developmental abnormalities, according to one or more embodiments.

FIG. 5 shows ethyl pyruvate prevented increased anterior chamber depth and reduced anterior chamber deepening, according to one or more embodiments.

FIG. 6 shows ethyl pyruvate in combination with additional therapeutic agents afforded increased disease protection, according to one or more embodiments.

FIG. 7 shows sodium pyruvate did not impact IOP elevation or developmental abnormalities.

FIG. 8 shows ethyl pyruvate alone, nicotinamide alone as well as the combination of ethyl pyruvate and nicotinamide protects from glaucomatous optic nerve damage, according to one or more embodiments.

FIGS. 9A and 9C are 3D fluorescent images of tissue volumes for trabecular meshwork and Schlemm's canal, respectively. FIGS. 9B and 9D are box plots for the corresponding data showing that the combination of ethyl pyruvate and nicotinamide rescues developmental anomalies of the ocular drainage tissues, according to one or more embodiments.

FIG. 10 shows that treatment of mice harboring an early onset Lmx1b developmental glaucoma model with ethyl pyruvate (2000 mg/kg/day) and NAM (550 mg/kg/day) profoundly prevented cell death.

### DETAILED DESCRIPTION

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

An “effective amount”, “sufficient amount” or “therapeutically effective amount” as used herein is an amount of a compound that is sufficient to effect beneficial or desired results, including clinical results. As such, the effective amount may be sufficient, for example, to reduce or ameliorate the severity and/or duration of an affliction or condition, or one or more symptoms thereof, prevent the advancement of conditions related to an affliction or condition, prevent the recurrence, development, or onset of one or more symptoms associated with an affliction or condition, or enhance or otherwise improve the prophylactic or therapeutic effect(s) of another therapy. An effective amount also includes the amount of the compound that avoids or substantially attenuates undesirable side effects.

The present application in accordance with one aspect, is directed to a method of treating or preventing a condition in a subject, wherein the method includes administering to the subject a therapeutically effective amount of ethyl pyruvate, or a derivative or analog thereof, or a pharmaceutical composition thereof. In accordance with some aspects the condition is selected from the group consisting of an ocular disease or disorder, a neurodegenerative disease or disorder, a vascular disease or disorder, a metabolic disease or disorder, an inflammatory disease or disorder, a disease or disorder involving abnormal cell death or oxidation, a disease or disorder linked to one or more Lmx1b mutations, and a combination thereof.

As used herein, the term “glaucoma” refers to an eye disease that results in damage to the retina and optic nerve and visual dysfunction or vision loss. Glaucoma occurs more commonly among older people. Vision loss from glaucoma is permanent and is irreversible. Glaucoma is typically associated with elevated intraocular pressure but glaucoma also includes normal tension glaucoma (NTG), which is a form of primary open angle glaucoma (POAG). Glaucoma may also be the result of traumatic neural injury.

As used herein, the term “normal intraocular pressure” (normal IOP) in humans refers to a human subject having an IOP value of 10 mmHg to 21 mmHg. Some individuals, however, may develop optic nerve damage despite a normal IOP (known as normal-tension glaucoma).

As used herein, the term “high intraocular pressure” (high IOP) in humans refers to a human subject having an IOP value greater than 21 mmHg (or 2.8 kPa). High IOP is known to be a risk factor for glaucoma. Some individuals, however, may have high IOP for years and never develop glaucoma or optic nerve damage.

As used herein, the term “preventing” or “prevention” with respect to, for example, neuronal damage or death in general or intraocular pressure in particular, refers to the ability of the compounds or agents of the present invention to confer neuroprotection, preferably before such damage, death, or disease occurs or to prevent a subject developing high intraocular pressure. Thus, prevention of high intraocular pressure includes avoiding the development of high intraocular pressure, reducing the risk or chance of eventually developing high intraocular pressure, delaying the onset or progression of high intraocular pressure, or reducing the severity of neuronal damage/extent of neuronal death/loss among a population of neurons should high intraocular pressure eventually develop.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease, a stabilized (i.e., not worsening) state of disease, preventing spread of disease, delay or slowing of disease progression, amelioration or palliation of the disease state and remission (whether partial or total), whether detectable or undetectable.

In some embodiments, the condition is an ocular disease or disorder. In some embodiments, the ocular disease or disorder comprises elevated intraocular pressure, one or more changes to the structure or function of one or more ocular tissues, abnormal cell death, or a combination thereof. In some embodiments, the one or more changes to the structure or function of one or more ocular tissues comprise malformation or dysfunction of ocular drainage structures, one or more developmental anomalies, neural or non-neural cell degeneration, dysfunction, or death, or a combination thereof. In some embodiments, the one or more changes to the structure or function of one or more ocular tissues result from environmental exposure, disease, aging, metabolic anomaly, mitochondrial anomaly, genetic variant or epigenetic differences, or a combination thereof. In some embodiments, the one or more developmental anomalies comprise changes to anterior chamber depth, pupil abnormalities, iridocorneal adhesions, trabecular meshwork, Schlemm's canal, cornea or a combination thereof. In some embodiments, the neural or non-neural cell degeneration or dysfunction comprises neurodegeneration and/or neural dysfunction in the subject's retinal ganglion cells. In some embodiments, malformation or malfunction of the ocular drainage tissues includes Schlemm's canal, trabecular meshwork, or a combination thereof.

In some embodiments, the ocular disease or disorder is a neurodegenerative disease or disorder. In some embodiments, the ocular disease or disorder is glaucoma.

In some embodiments, the condition is a neurodegenerative disease not directly affecting the ocular system. In some embodiments, the condition comprises late onset neurodegeneration. In some embodiments, the condition is Alzheimer's disease or Parkinson's disease.

In some embodiments, the disease or disorder linked to one or more Lmx1b mutations is a disease or disorder of the brain or kidney. In some embodiments, the one or more Lmx1b variants lead to cell stress, developmental anomalies, cell death, and/or elevated IOP, which in turn may lead to a further disease or condition. In some embodiments, Lmx1b mutations cause developmental defects, organ disease (e.g., kidney disease), ocular disease (e.g., glaucoma), and/or neurodegenerative disease (e.g., Alzheimer's disease and Parkinson's disease). In some embodiments, Lmx1b-associated diseases or conditions present with different ages of onset.

In some embodiments, the condition is age-related macular degeneration. In some embodiments, the condition is traumatic glaucoma.

In some embodiments, the condition comprises a retinal pigment epithelium.

In some embodiments, the method further includes administering one or more additional therapeutic agents, or one or more pharmaceutical compositions thereof, to the subject. In some embodiments, the one or more additional therapeutic agents are selected from the group consisting of an antioxidant agent, an anti-inflammatory agent, an agent that modulates metabolism, an agent that modulates the integrated stress response, an agent that modulates the unfolded protein response, an agent that modulates forms of autophagy, an agent that modulates the expression or activity of genes controlling or mediating antioxidant or other protective responses, a senolytic agent, an agent that modulates the mitochondria or mitophagy, an anti-aging agent, an agent that modulates intraocular pressure, a resilience-boosting agent, an antifibrotic agent, an agent that prevents epithelial mesenchymal transition or endothelial mesenchymal transition, a neuroprotective agent, a gene therapy agent, and a combination thereof. In some embodiments, the agent modulates metabolism reprograms or boosts metabolism. In some embodiments, the antioxidant agent reduces oxidative stress and boosts antioxidant control. In some embodiments, the gene therapy agent results in genome editing, genome reprogramming, epigenetic editing, epigenetic reprogramming, or a combination thereof. In some embodiments, the agent that prevents epithelial mesenchymal transition or endothelial mesenchymal transition is an anti-transforming growth factor-β (“TGFB”) or ligand trap molecule. In some embodiments, the one or more additional therapeutic agents are selected from the group consisting of nicotinamide (NAM), nicotinamide mononucleotide, nicotinamide adenine dinucleotide, nicotinamide ribose, pyrroloquinoline quinine, N-acetyl cysteine, and a combination thereof.

Examples of other agents that may be used in combination with ethyl pyruvate include, but are not limited to, saffron, berry extract or powder, antioxidants (e.g., billberry), flavonoids, anthocyanins, carotenoids, polyphenols, folate, xcoxanthine, polyamines (e.g., spermidine), Ginko biloba (glaucoma vitamin supplement (Glaucocetin), Co enzyme Q10, Vitamin A, Vitamin B12, Vitamin B50 complex, Vitamin C, Vitamin D, Vitamin E, betaine, choline, citicholine, Epigallocatechin-3-gallate (EGCG and its derivatives or analogs), compounds that increase activity of Sirts including Sirt3, lutein, xeozanthine, molecules that induce genes that protect from oxidative damage, dimethylfumarate and other forms of fumarate, and antifibrotic agents (such as anti-TGFBs, curcumin, blueberry, silymarin, coffee, vitamin C, E, and D, resveratrol, quercetin, and epigallocatechin-3-gallate). In accordance with some embodiments, the additional agent may be a metabolism supporting molecule, such as, but not limited to: ketones, ketone bodies-metabolites used in energy metabolism (e.g., hydroxubutarate, Beta-hydroxybutarate (BHB) and its salts, e.g., acetoacetate. The methods disclosed herein may also be combined with diets shown to help in glaucoma such as, but not limited to, ketogenic diet and low carb diet. The methods disclosed herein may also be combined with glucagon like peptide (GLP-1) agonists including, but not limited to, Exenatide, Liraglutide and Semaglutide. Taurine and creatine can also be used in combination with the methods disclosed herein. Additional examples useful herein also include molecules and gene therapies that increase or induce NMNAT2 (nicotinamide mononucleotide adenosyl transferase) or other NMNATs, molecules or gene therapies that induce or otherwise increase signaling of the TEK/Angiopoietin system, any molecules that increase NAD, or insulin and its derivatives, agents or gene therapies that increase Ca²⁺/calmodulin-dependent protein kinase II (CAMKII) activity/signaling, treatments that increase CNTF, BNDF or other beneficial growth factors. This also includes without limitation encapsulated cells in implants that release the therapeutic agent(s) including these growth factors and/or proteins, peptides, antibodies, agonists, anti-inflammatory molecules, and metabolites. This also includes similar systems for ANPT/TEK, insulin, etc. Additional examples that can be combined with EP treatment include any IOP lowering medications e.g. Latanprost and other prostaglandin analogs. The method disclosed herein could also be combined with laser and/or other glaucoma surgeries aimed at lowering IOP, with various implanted tubes, shunts and stents that lower IOP or long-term drug formulations and delivery devices. Biotin, hemp seeds/powder, long chain polyunsaturated fatty acids (PUFAS) (e.g., omega 3 fatty acids), spirulina and leafy green powder or extracts can also be combined with ethyl pyruvate treatment as disclosed herein. Derivatives and analogs of the compounds disclosed herein can also be used.

In some embodiments, the one or more additional therapeutic agents are administered together in a pharmaceutical composition with ethyl pyruvate. In other embodiments, the one or more additional therapeutic agents are administered separately from ethyl pyruvate. In still other embodiments, the subject is already being treated with the one or more additional therapeutic agents when the ethyl pyruvate is administered; or the subject is administered the one or more additional therapeutic agents after being administered the ethyl pyruvate.

In some embodiments, the ethyl pyruvate is administered in an amount sufficient to treat or prevent the condition in the subject. In some embodiments, the ethyl pyruvate is administered in an amount sufficient to prevent or reduce elevated intraocular pressure, prevent or reduce one or more changes to the structure or function of one or more ocular tissues, prevent or reduce abnormal cell death, or a combination thereof. In some embodiments, the ethyl pyruvate is administered in an amount sufficient to prevent or reduce malformation or dysfunction of ocular drainage structures, prevent or reduce one or more developmental anomalies, prevent or reduce neural or non-neural cell degeneration, dysfunction, or death, or a combination thereof. In some embodiments, the ethyl pyruvate is administered in an amount sufficient to prevent or reduce one or more changes in the subject resulting from environmental exposure, disease, aging, metabolic anomaly, mitochondrial anomaly, genetic mutation, or a combination thereof. In some embodiments, the ethyl pyruvate is administered in an amount sufficient to prevent or reduce one or more developmental anomalies in the subject, such as, but not limited to, changes to anterior chamber depth, pupil abnormalities, iridocorneal adhesions, trabecular meshwork, Schlemm's canal, cornea or a combination thereof. In some embodiments, the ethyl pyruvate is administered in an amount sufficient to prevent or reduce cell degeneration or dysfunction in the subject, such as, but not limited to, neurodegeneration and/or neural dysfunction in the subject's retinal ganglion cells. In some embodiments, the ethyl pyruvate is administered in an amount sufficient to prevent or reduce changes or dysfunction to the subject's ocular drainage structures, such as, but not limited to, Schlemm's canal, trabecular meshwork, or a combination thereof. In some embodiments, the ethyl pyruvate is administered in an amount sufficient to prevent or reduce a neurodegenerative disease or disorder in the subject, such as, but not limited to, glaucoma or a late onset neurodegeneration (e.g., Alzheimer's disease or Parkinson's disease). In some embodiments, the ethyl pyruvate is administered in an amount sufficient to prevent or reduce disease or disorder linked to one or more Lmx1b mutations, such as, but not limited to, a disease or disorder of the eye, brain, or kidney. In some embodiments, the ethyl pyruvate is administered in an amount sufficient to prevent or reduce a condition of aging in the subject, such as, but not limited to, age-related macular degeneration. In some embodiments, the ethyl pyruvate is administered in an amount sufficient to prevent or reduce a retinal pigment epithelium in the subject.

In some embodiments, the amount of ethyl pyruvate sufficient to produce the effects described in the preceding paragraph will be the same when administered alone or in combination with one or more additional therapeutic agents. In some embodiments, the amount of ethyl pyruvate sufficient to produce the effects described in the preceding paragraph will be less when administered with one or more additional therapeutic agents than when administered alone.

In some embodiments, the subject is a mammal. In some embodiments, the subject is a mouse or rat. In some embodiments, the subject is of canine or equine origin. In some embodiments, the subject is a non-human primate. In some embodiments, the subject is a human.

In some embodiments, the subject has the condition. In other embodiments, the subject is at risk, including, but not limited to, genetic risk, for developing the condition. In other embodiments, the subject has the condition and is at risk, including, but not limited to, genetic risk, for developing more serious variants or complications of the condition. In some embodiments, the subject has the condition and is at risk, including, but not limited to, genetic risk, for developing additional conditions, which additional conditions may or may not be related to the original condition.

In another aspect, a method of reducing or preventing elevated intraocular pressure is described, the method comprising administering a therapeutically effective amount of ethyl pyruvate, or a pharmaceutical composition thereof, to the subject.

In another aspect, a method of reducing or preventing damage or dysfunction to a subject's eye tissues is described, the method comprising administering a therapeutically effective amount of ethyl pyruvate, or a pharmaceutical composition thereof, to the subject.

In another aspect, a method of reducing or preventing damage or dysfunction to tissues involved in ocular fluid drainage in a subject is described, the method comprising administering a therapeutically effective amount of ethyl pyruvate, or a pharmaceutical composition thereof, to the subject.

In another aspect, a method of reducing or preventing developmental malformations or dysfunctions in a subject's eye is described, the method comprising administering a therapeutically effective amount of ethyl pyruvate, or a pharmaceutical composition thereof, to the subject.

In any one of the embodiments described herein, the method further comprises administering one or more additional therapeutic agents, or one or more pharmaceutical compositions thereof, to the subject.

In some embodiments, the ethyl pyruvate is administered in an amount sufficient to reduce intraocular pressure in the subject or to prevent the subject's intraocular pressure from increasing. In some embodiments, the subject has elevated intraocular pressure and the administration of ethyl pyruvate reduces the intraocular pressure or prevents the intraocular pressure from further increasing. In some embodiments, the subject does not have elevated intraocular pressure, and the administration of ethyl pyruvate prevents the subject's intraocular pressure from increasing.

In some embodiments, the ethyl pyruvate is administered in an amount sufficient to prevent or reduce neurodegeneration and/or neural dysfunction in the subject's neural and non-neural cells. In some embodiments, the subject has the neurodegeneration and/or neural dysfunction and the administration of ethyl pyruvate reduces the neurodegeneration and/or neural dysfunction or prevents further neurodegeneration and/or neural dysfunction. In other embodiments, the subject does not have the neurodegeneration and/or neural dysfunction and the administration of ethyl pyruvate prevents the neurodegeneration and/or neural dysfunction from developing. In some embodiments, the effects of ethyl pyruvate for preventing or reducing neurodegeneration and/or neural dysfunction are related to its activity for modulation of intraocular pressure. In other embodiments, the effects of ethyl pyruvate for preventing or reducing neurodegeneration and/or neural dysfunction are independent of its activity for modulating intraocular pressure.

In some embodiments, the ethyl pyruvate is administered in an amount sufficient to prevent or reduce changes in the subject's ocular drainage tissues. In some embodiments, the subject has the changes in ocular drainage tissues and the administration of ethyl pyruvate reduces the changes or prevents further changes. In other embodiments, the subject does not have the changes and the administration of ethyl pyruvate prevents the changes from developing. In some embodiments, the effects of ethyl pyruvate for preventing or reducing changes in the subject's ocular drainage tissues are related to its activity for modulation of intraocular pressure. In other embodiments, the effects of ethyl pyruvate for preventing or reducing changes in the subject's ocular drainage tissues are independent of its activity for modulating intraocular pressure.

In some embodiments, the ethyl pyruvate is administered in an amount sufficient to prevent or reduce one or more dysfunctions and/or developmental changes in the subject's eye. In some embodiments, the one or more dysfunctions and/or developmental changes are dysfunctions and/or malformations of the subject's ocular drainage tissues, including, but not limited to, Schlemm's canal and trabecular meshwork. In some embodiments, the one or more dysfunctions and/or developmental changes are anterior chamber depth, pupil abnormalities, trabecular meshwork, Schlemm's canal, and/or iridocorneal adhesions. In some embodiments, the subject has the dysfunctions and/or developmental changes and the administration of ethyl pyruvate reduces the developmental changes or prevents further developmental changes. In other embodiments, the subject does not have the dysfunctions and/or developmental changes and the administration of ethyl pyruvate prevents the developmental changes from developing. In some embodiments, the effects of ethyl pyruvate for preventing or reducing one or more dysfunctions and/or developmental changes in the subject's eye are related to its activity for modulation of intraocular pressure. In other embodiments, the effects of ethyl pyruvate for preventing or reducing one or more dysfunctions and/or developmental changes in the subject's eye are independent of its activity for modulating intraocular pressure.

In some embodiments, the disease or disorder of the eye is a neurodegenerative disease. In some embodiments, the disease or disorder of the eye is glaucoma.

In some embodiments, the disease or disorder of the eye is a non-neurodegenerative disease.

In some embodiments, the disease or disorder of the eye is an age-related disease or disorder. In some embodiments, the disease of disorder of the eye is age-related macular degeneration. In some embodiments, age-related macular degeneration includes neurodegeneration. In some embodiments, age-related macular degeneration includes a retinal pigment epithelium (“RPE”). In some embodiments, age-related macular degeneration includes a vascular disease or condition.

In some embodiments, the prevention or reduction of the subject's elevated intraocular pressure prevents or treats a disease or disorder of the eye. In some embodiments, the disease or disorder of the eye comprises non-neural cell degeneration, dysfunction, and/or death. In some embodiments, the disease or disorder of the eye comprises neural cell degeneration, dysfunction, and/or death. In some embodiments, the disease or disorder of the eye is a neurodegenerative disease. In some embodiments, the disease or disorder of the eye is glaucoma. In some embodiments, the disease or disorder of the eye is a non-neurodegenerative disease. In some embodiments, the disease or disorder of the eye is an age-related disease or disorder. In some embodiments, the disease of disorder of the eye is age-related macular degeneration. In some embodiments, the disease or disorder of the eye is glaucoma or age-related macular degeneration.

In some embodiments, the prevention or reduction of the subject's elevated intraocular pressure prevents or treats neurodegeneration and/or neural dysfunction in the subject's retinal ganglion cells, changes in the subject's ocular drainage tissues, and/or developmental changes in the subject's eye.

Ethyl pyruvate has the chemical structure

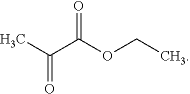

The present disclosure also contemplates derivatives or analogs of ethyl pyruvate. Derivatives or analogs of ethyl pyruvate may include, but are not limited to, different ester chain lengths or substitutions, different substitutions on the acyl carbon, halogenated and isotopically-derived analogs, and isomers and combinations thereof; and pharmaceutically acceptable salts thereof. Derivatives or analogs of ethyl pyruvate may also include, but are not limited to, conjugates with imaging agents, macromolecules, biomacromolecules, targeting agents, and isomers and combinations thereof; and pharmaceutically acceptable salts thereof. Use of ethyl pyruvate or its derivatives and analogs in a pharmaceutical composition with one or more pharmaceutically acceptable excipients, and/or with one or more drug delivery or targeting vehicles, are also contemplated.

In accordance with some embodiments, the derivative or analog of ethyl pyruvate is selected from the group consisting of methyl pyruvate, propyl pyruvate, butyl pyruvate, pentyl pyruvate, hexyl pyruvate, octyl pyruvate, isobutyl pyruvate, isopentyl pyruvate, isoheptyl pyrvate, isooctyl pyruvate, cyclopentyl pyruvate, cyclopentylmethyl pyruvate, cyclohexyl pyruvate, cyclohexylmethyl pyruvate, butenyl pyruvate, hexenyl pyruvate, isobutenyl pyruvate, isohexenyl pyruvate, butinyl pyruvate, hexinyl pyruvate, methoxymethyl pyruvate, ethoxymethyl pyruvate, ethoxycarbonylmethyl pyruvate and a combination thereof.

In some embodiments, elevated IOP is a risk factor for a disease or disorder. In some embodiments, elevated IOP is a risk factor for a disease or disorder of the eye. In some embodiments, elevated IOP is a risk factor for a non-neurodegenerative or a neurodegenerative disease or disorder of the eye. In some embodiments, elevated IOP is a risk factor for glaucoma. In some embodiments, elevated IOP is a risk factor for dysfunctions, malformations, and/or death of one or more cells, tissues, or structures in the eye.

In some embodiments, ethyl pyruvate protects ocular tissues from stresses due to environment, development, disease, and/or age (including, but not limited to, mitochondrial and metabolic abnormalities, Lmx1b mutation, or mutations in other genes impacting metabolism, development, and/or glaucoma.

In some embodiments, ethyl pyruvate protects ocular drainage structures from changes and cell death due to developmental conditions, genetic and epigenetic effects, and/or environmental and/or exposure effects. In some embodiments, ethyl pyruvate protects ocular drainage structures from malformation. In some embodiments, ethyl pyruvate protects from developmental anomalies of the eye. In some embodiments, ethyl pyruvate protects from developmental malformations that may have functional, cosmetic, and/or psychological impacts on the subject.

In some embodiments, ethyl pyruvate protects from cell death in ocular tissues and/or IOP elevation. In some embodiments, this cell death and IOP elevation is independent from glaucomatous neurodegeneration, while, in other embodiments, it is linked to glaucomatous neurodegeneration. Therefore, in certain embodiments, ethyl pyruvate protects from glaucomatous and other ocular neurodegenerations.

In some embodiments, ethyl pyruvate protects from dysfunction and death of cells. In some embodiments, these cells may be nerve cells and/or neurons.

In some embodiments, ethyl pyruvate protects from diseases or disorders of the brain, including, but not limited to, late onset neurodegeneration (e.g., Parkinson's disease and Alzheimer's disease).

In some embodiments, ethyl pyruvate protects from diseases or disorders associated with aging.

In some embodiments, ethyl pyruvate, either alone or in combination with other therapeutic agents (e.g., nicotinamide), is an effective treatment for early-onset glaucoma with developmental abnormalities. In some embodiments, ethyl pyruvate, either alone or in combination with other therapeutic agents (e.g., nicotinamide), is an effective treatment for later-onset glaucomas.

In some embodiments, the inventors surprisingly found that ethyl pyruvate protects mice with a Lmx1b mutation. In some embodiments, Lmx1b mutations cause developmental defects, kidney disease, and/or glaucoma, with different ages of onset in mice versus humans. Therefore, in some embodiments, ethyl pyruvate treatment may protect from various Lmx1b-induced diseases.

In some embodiments, the inventors surprisingly found that ethyl pyruvate lessens IOP elevation in mouse models comprising a genetic model resulting in cell stress, developmental anomalies, cell death, and/or high IOP. In some embodiments, one or more of these factors may lead to glaucoma. In some embodiments, the mouse model is a Lmx1b mouse model. This is unexpected at least because sodium pyruvate had no effect on IOP in age-onset mouse glaucoma models, or in the early-onset Lmx1b developmental glaucoma model. For example, treatment of Lmx1b mice with about 500 mg/kg/day of sodium pyruvate had no effect on IOP at 2, 3, and 4-months post-treatment. This is also unexpected at least because calcium pyruvate had no effect on IOP in humans in prior clinical studies.

In some embodiments, the inventors surprisingly found that ethyl pyruvate reduces the presence and severity of developmental abnormalities of anterior eye structures in mouse models of glaucoma. This is unexpected at least because sodium pyruvate treatment showed no change in the severity of anterior eye developmental abnormalities. For example, treatment of Lmx1b mice with about 500 mg/kg/day of sodium pyruvate had no effect on the severity of anterior eye developmental abnormalities at 2, 3, and 4-months post-treatment.

In some embodiments, dosages of about 2000 mg/kg/day ethyl pyruvate lessen the severity of ocular developmental abnormalities and IOP elevation in mice. In some embodiments, combination of ethyl pyruvate and other agents capable of modulating metabolism (e.g., metabolic boosting agents) affords a higher degree of protection against the severity of ocular developmental abnormalities and IOP elevation in mice compared to ethyl pyruvate alone. For example, in some embodiments, combination of ethyl pyruvate and a metabolic boosting agent affords greater protection against developmental pupil abnormalities.

In some embodiments, the inventors surprisingly found that ethyl pyruvate is more protective than sodium pyruvate. In some embodiments, the inventors surprisingly found that ethyl pyruvate is a superior neuroprotectant than sodium pyruvate.

In some embodiments, ethyl pyruvate enables use of lower dosages of other treatment modalities, such as, but not limited to, nicotinamide and other agents capable of modulating metabolism.

Without wishing to be bound by theory, the superior properties of ethyl pyruvate as compared to existing treatments (e.g., sodium pyruvate) may stem from ethyl pyruvate's enhanced stability, cell penetration properties, antioxidant properties, anti-inflammatory properties, anti-apoptotic properties, and/or metabolic (bioenergetic) properties (e.g., relative to sodium pyruvate). Again, not wishing to be bound by theory, the enhanced anti-inflammatory and anti-apoptotic properties of ethyl pyruvate may result from its molecular structure, stability, and/or enhanced ability to permeate into cells. See, e.g., Chenxi Lu et al, Ethyl Pyruvate: A Newly Discovered Compound Against Ischemia-Reperfusion Injury in Multiple Organs, 171 Pharmacological Res. 105757 (2021); Ivan Koprivica, Ethyl Pyruvate, A Versatile Protector in Inflammation and Autoimmunity, 71 Inflammation Res. 169 (2022).

### Pharmaceutical Compositions

This application also provides a pharmaceutical composition comprising at least one of the compounds as described herein or a pharmaceutically-acceptable salt thereof, and a pharmaceutically-acceptable carrier.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and pharmaceutically acceptable for the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As set out above, certain embodiments of the present pharmaceutical agents may be provided in the form of pharmaceutically-acceptable salts. The term “pharmaceutically-acceptable salt”, in this respect, refers to the relatively non-toxic, inorganic and organic acid addition salts of compounds of the present application.

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), and/or parenteral administration. Particularly useful formulations include oral and direct to eye or locally around the eye formulations. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets, may be, made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein.

Liquid dosage forms for oral administration of the compounds disclosed herein include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives or buffers which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances.

Transdermal patches have the added advantage of providing controlled delivery of a compound of the present invention to the body. Such dosage forms can be made by dissolving or dispersing the pharmaceutical agents in the medium. Absorption enhancers can also be used to increase the flux of the pharmaceutical agents of the invention across the skin. The rate of such flux can be controlled, by either providing a rate controlling membrane or dispersing the compound in a polymer matrix or gel.

Ophthalmic formulations, eye ointments, powders, solutions and the like, are also contemplated as being within the scope of this disclosure. Implants or injectables may also be used to introduce the agent into the eye. Any of the excipients disclosed herein suitable for use in these applications can be incorporated into the pharmaceutical formulations.

Pharmaceutical compositions of this invention suitable for parenteral administration comprise one or more compounds disclosed herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

In some embodiments, dosages of from about 100-2000, more particularly from about 250-1000 mg/kg/day ethyl pyruvate lessen the severity of ocular developmental abnormalities and IOP elevation in mice. Human equivalent doses can be calculated as described in the literature. In some embodiments, combination of ethyl pyruvate and other agents capable of modulating metabolism (e.g., metabolic boosting agents) affords a higher degree of protection against the severity of ocular developmental abnormalities and IOP elevation in mice compared to ethyl pyruvate alone. For example, in some embodiments, combination of ethyl pyruvate and NAM affords greater protection against developmental pupil abnormalities. In some embodiments, such as in the case of traumatic injury to optic nerve, eye or neural tissue and short term treatment a dose of up to about 8000 mg/kg/day in mice could be used. Again, the human equivalent dose can be calculated accordingly.

In some embodiments, a combination of ethyl pyruvate and NAM are particularly useful. In some embodiments, the dosage of NAM is from about 100-2500, more particularly from about 500-1000 mg/kg/day. Human equivalent doses can be calculated as described in the literature. Nicotinamide riboside could also be used at similar or lower doses.

One of ordinary skill in the art can convert the dosages from one species to another using the teachings in Freireich et al., Quantitative comparison of toxicity of anticancer agents in mouse, rat, dog, monkey and man, Cancer Chemother Rep. 50 (4): 219-244, 1966 (incorporated herein by reference) or similar methods to calculate species specific equivalent doses. Nair A B, Jacob S. A simple practice guide for dose conversion between animals and human. J Basic Clin Pharm. 2016; 7:27-31. This results in an animal equivalent dosage based on the mouse dosage. For example, a mouse dosage of 550 mg/kg/day ethyl pyruvate is equivalent to a dose of about 2.7-3 g/day for a 70 kg person. In some embodiments, treatment is continued until a certain target milestone is obtained. In some cases, treatment is continued for an extended period of time, such as weeks, months or years to prevent development of glaucoma and/or elevated IOP.

In accordance with some embodiments, typical dosing may be once, twice, three or more times a day. Total daily dose may be administered once, or administered as two, three or more separate doses. For multiple dosing, each dose can be the same amount or different amounts. The pharmaceutical composition may be administered in the morning or evening. The pharmaceutical composition may be taken with or without meals.

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

### Example 1: Ethyl Pyruvate Provides Disease Protection

Ethyl pyruvate showed a protective effect in mice with a Lmx1b mutation, thereby demonstrating that ethyl pyruvate treatment can protect from various Lmx1b-induced diseases. For example, Lmx1b is implicated in neurodegenerative conditions like glaucoma and Parkinson's disease. Genetic models having a Lmx1b mutation may result in cell stress, developmental anomalies, cell death, and/or elevated IOP, which can lead to further diseases or conditions. For example, ethyl pyruvate lessened IOP and rescued ocular developmental abnormalities in mouse models of glaucoma, across multiple dosages and with or without co-administration of another therapeutic agent.

Ethyl pyruvate was sourced from Sigma-Aldrich. Ethyl pyruvate has been shown to be safe and well-tolerated across multiple mammalian species, including humans, mice, rats, and horses. See, e.g., Clinical Trial Identifier NCT00107666 (human cardiac trial, dosing ethyl pyruvate at 7.5 g (90 mg/kg) intravenously, every six hours for two days); Xiujing Sun et al., Ethyl Pyruvate Supplemented in Drinking Water Ameliorates Experimental Nonalcoholic Steatohepatitis, 137 Biomedical Pharmacotherapy 111392 (2021); Leilei Mao et al., Ethyl Pyruvate Improves White Matter Remodeling in Rats After Traumatic Brain Injury, 27 CNS Neuroscience & Therapeutics 113 (2021); C. C. Jacobs et al, Ethyl Pyruvate Diminishes the Inflammatory Response to Lipopolysaccharide Infusion in Horses, 45 Equine Veterinary J. 333 (2013). Mice have been dosed for at least up to nine months without adverse effects. Furthermore, ethyl pyruvate is an FDA-approved food additive and flavoring agent.

A dose of about 2000 mg/kg ethyl pyruvate in mice is equivalent to about 9.8 g/day for a 60 kg human.

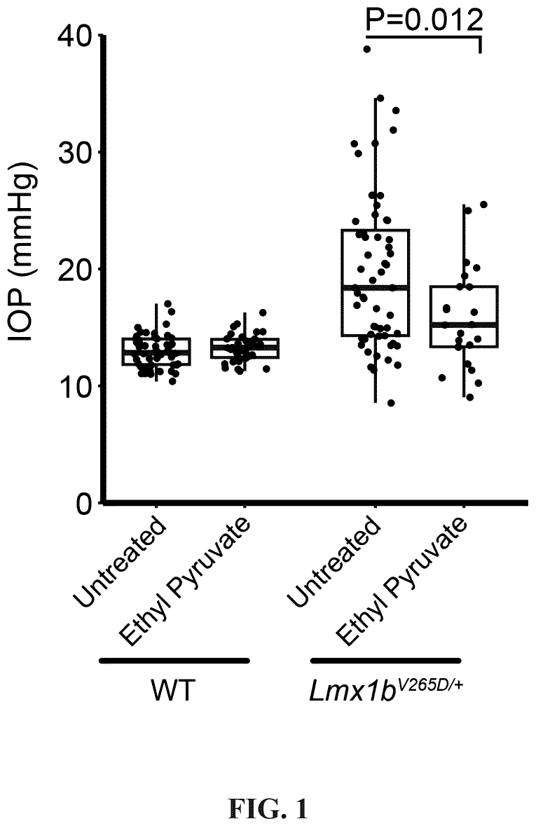

FIG. 1 shows that treatment of mice harboring an early-onset Lmx1b developmental glaucoma model with ethyl pyruvate (500 mg/kg/day) resulted in lessening of IOP out to two months of age (age assessed). This demonstrated significant difference showing that ethyl pyruvate protected against high IOP.

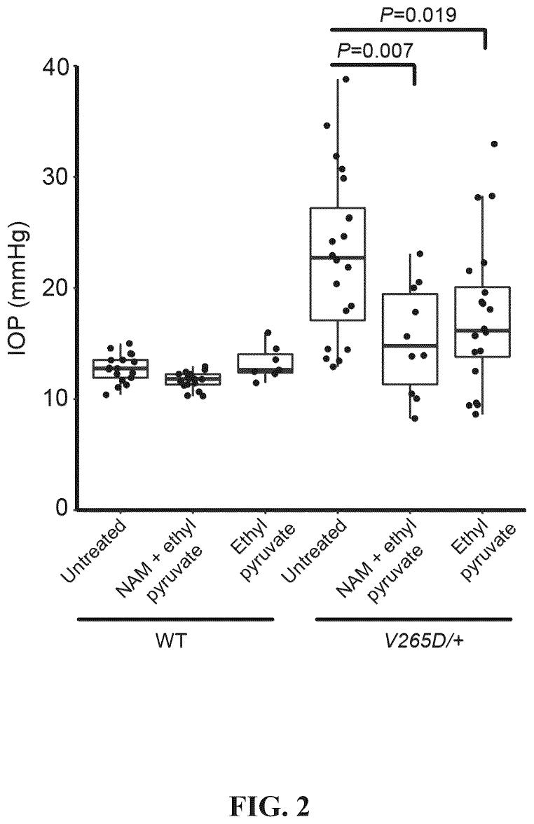

FIG. 2 shows that treatment of mice harboring an early onset Lmx1b developmental glaucoma model with ethyl pyruvate (2000 mg/kg/day) resulted in lessening of IOP. FIG. 2 further shows that treatment of mice harboring an early onset Imx1b developmental glaucoma model with ethyl pyruvate (500 mg/kg/day) and nicotinamide (550 mg/kg/day) was also protective from IOP elevation.

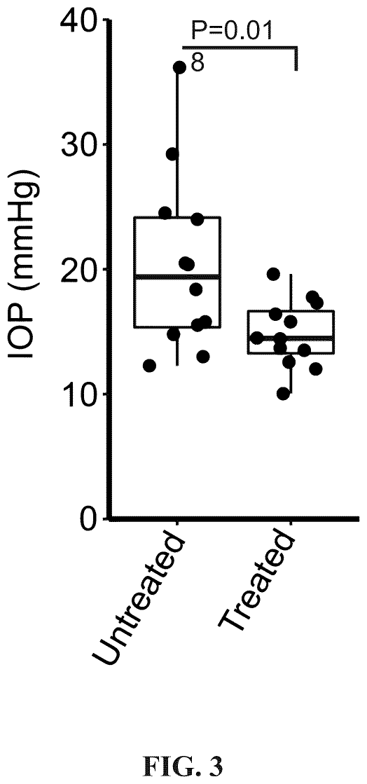

As shown in FIG. 3, combination of ethyl pyruvate with nicotinamide and other metabolic boosting agents had a higher degree of protection against the severity of IOP elevation. FIG. 3 shows that treatment of mice harboring an early onset ILmx1b developmental glaucoma model with ethyl pyruvate (2000 mg/kg/day), nicotinamide (550 mg/kg/day), and N-acetyl-L-cysteine (200 mg/kg/day) increased disease protection. Treatment began at P2 and IOP was measured at 5 weeks of age.

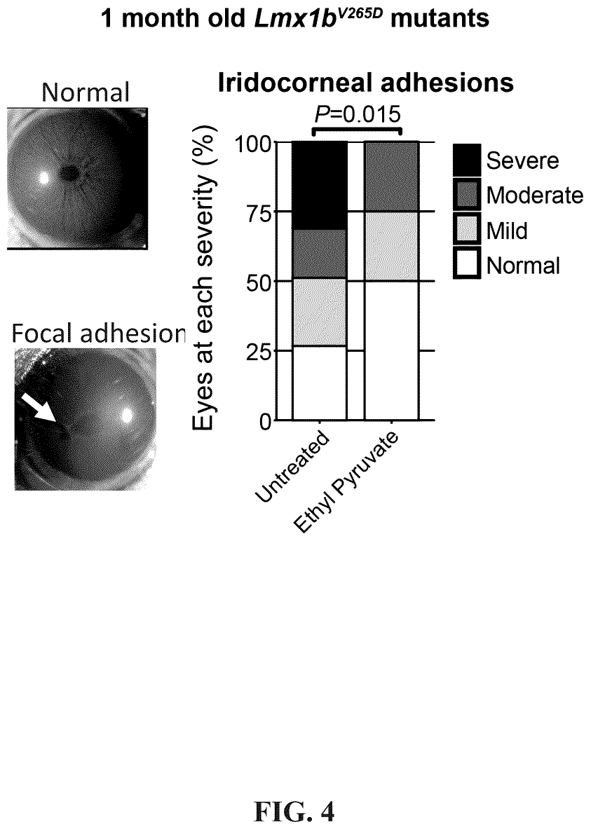

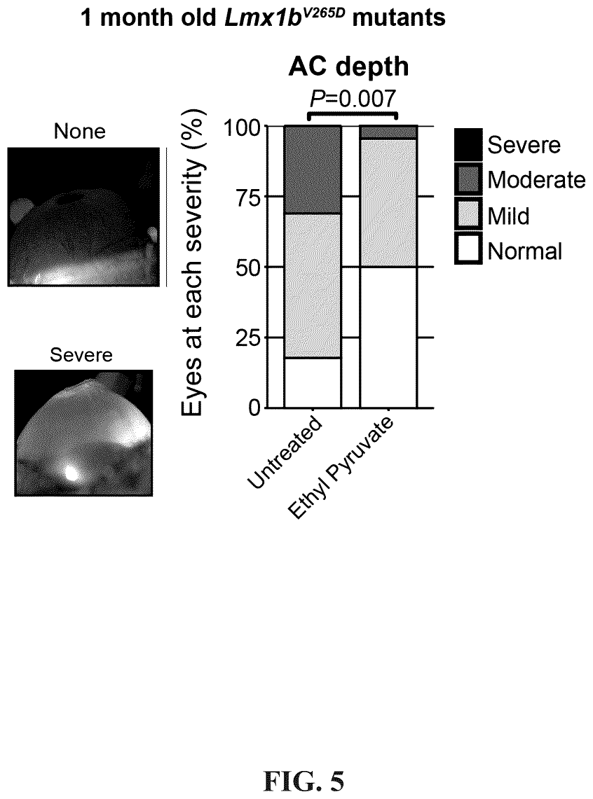

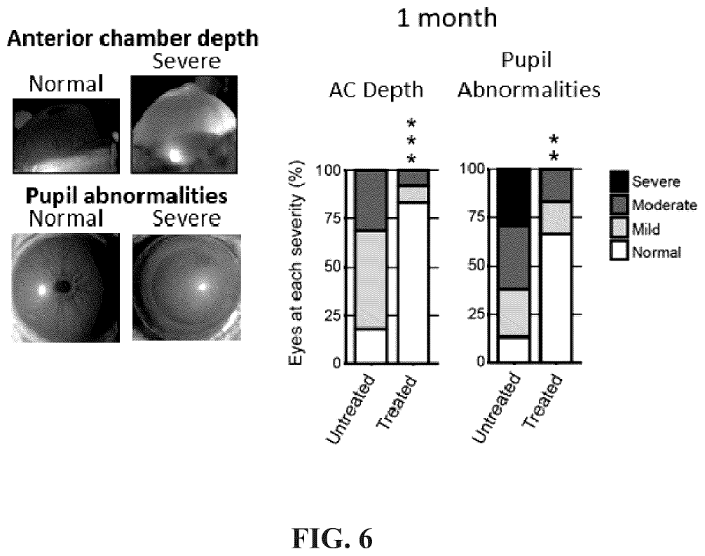

As shown in FIGS. 4-6, in addition to preventing or treating high IOP, treatment of mice harboring an early onset Lmx1b developmental glaucoma model with ethyl pyruvate, alone or in combination with a metabolite boosting agent, unexpectedly rescued ocular developmental abnormalities. FIG. 4 shows that ethyl pyruvate (2000 mg/kg/day) treatment showed less incidence and severity of abnormal attachments between the iris and the cornea (i.e., iridocorneal adhesions). Ethyl pyruvate treatment also showed a significant effect in preventing or reducing anterior chamber deepening, as shown in FIGS. 5-6. The anterior chamber can deepen in developmental glaucomas as a consequence of high IOP; therefore, this result was consistent with ethyl pyruvate protecting from high IOP.

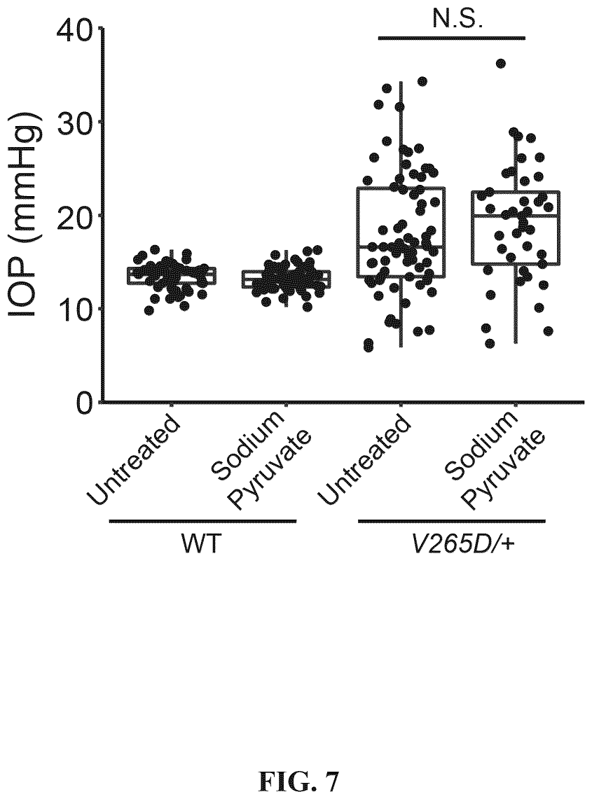

Ethyl pyruvate's ability to treat or prevent IOP elevation ocular developmental abnormalities was surprising at least because such effects have not been observed with sodium pyruvate treatments. FIG. 7 shows that treatment of mice harboring a glaucoma model with sodium pyruvate (500 mg/kg/day) had no effect on IOP elevation after two months. IOP was also measured at three and four months, and no changes were observed compared to untreated. Additionally, sodium pyruvate-treated mice showed no change in the severity of anterior eye developmental abnormalities.

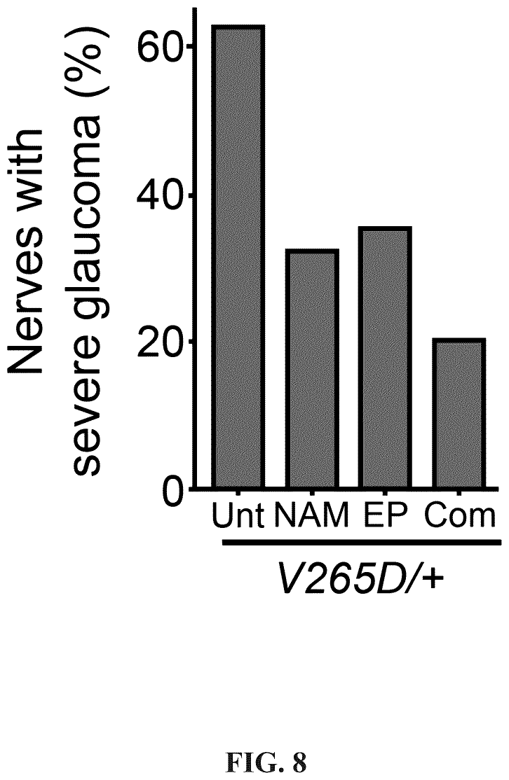

FIG. 8 shows that treatment of mice harboring an early onset Lmx1b developmental glaucoma model with ethyl pyruvate (2000 mg/kg/day) resulted in lessening of glaucomatous optic nerve damage (neurodegeneration) in mice aged to six months old. This shows an extension of the protection time period to a point where the majority of the untreated mice had severe glaucoma. FIG. 8 further shows that treatment of mice harboring an early onset Lmx1b developmental glaucoma model with nicotinamide (550 mg/kg/day) was also protective from glaucomatous optic nerve damage. FIG. 8 further shows that treatment of mice harboring an early onset Lmx1b developmental glaucoma model with ethyl pyruvate (2000 mg/kg/day) and nicotinamide (550 mg/kg/day) was also protective from glaucomatous optic nerve damage. As shown in the plotted data, the combined treatment provided the most protection against glaucomatous optic nerve damage.

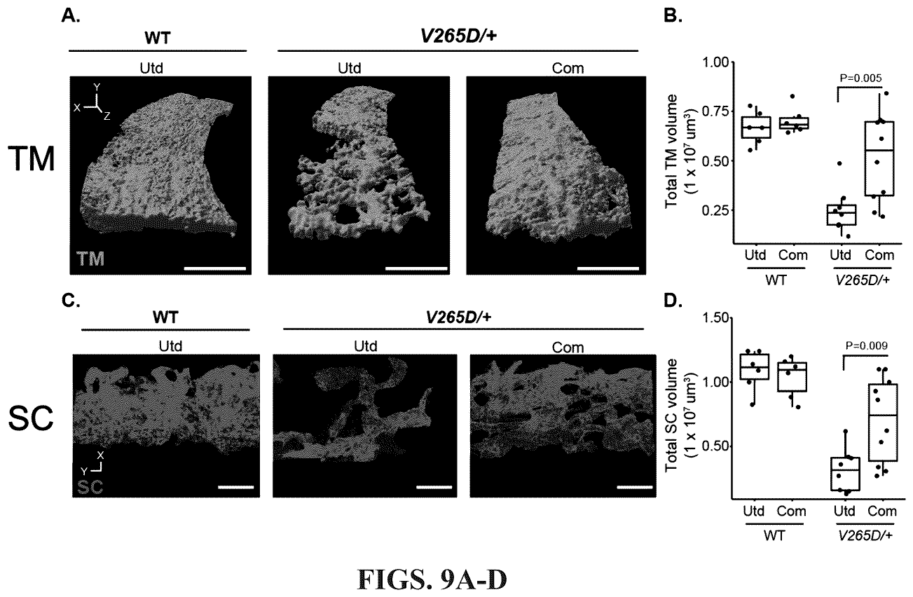

FIG. 9 shows that treatment of mice harboring an early onset Lmx1b developmental glaucoma model with ethyl pyruvate (2000 mg/kg/day) and NAM (500 mg/kg/day) administered to the mother rescued development anomalies of the ocular drainage tissues in the pup, which would also receive active from the mother's milk after birth for the first two to three weeks of life. This was a morphogenesis correction study (drainage tissue morphogenesis is complete by postnatal day 21 in mice). The mice were analyzed at 3 to 4 weeks of age. Treatment was started at birth in drinking water (prenatal administration to mother also works). FIGS. 9A and 9C show 3D representations of tissue volumes for TM=trabecular meshwork and SC=Schlemm's canal. FIGS. 9B and 9D are box plots for the corresponding data. (Abbreviations: UTD=untreated. Com=combined treatment).

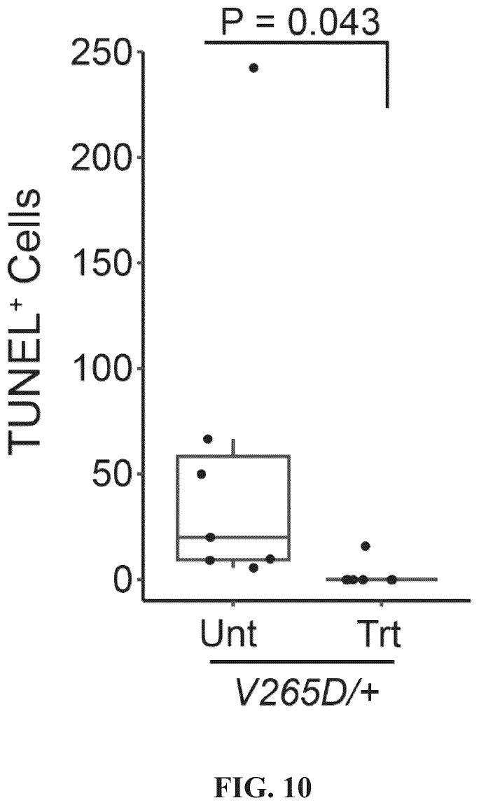

FIG. 10 shows that treatment of mice harboring an early onset Lmx1b developmental glaucoma model with ethyl pyruvate (2000 mg/kg/day) and NAM (550 mg/kg/day) profoundly prevented cell death. The subject mice treated from postnatal day 2 and assessed at postnatal day P10 an age where cell death is readily detected in this glaucoma model. Cell death was quantified as TUNEL+ cells in the TM per section. This was extrapolated to the entire TM volumes.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

What is claimed is:

1. A method of treating or preventing a condition in a subject, the method comprising administering to the subject a therapeutically effective amount of ethyl pyruvate, or a derivative or analog thereof, or a pharmaceutical composition thereof;
wherein the condition is selected from the group consisting of an ocular disease or disorder, a neurodegenerative disease or disorder, a vascular disease or disorder, a metabolic disease or disorder, an inflammatory disease or disorder, a disease or disorder involving abnormal cell death or oxidation, a disease or disorder linked to one or more Lmx1b mutations, and a combination thereof.

2. The method of claim 1, wherein the condition is an ocular disease or disorder.

3. The method of claim 1, wherein the ocular disease or disorder comprises elevated intraocular pressure, one or more changes to the structure or function of one or more ocular tissues, abnormal cell death, or a combination thereof.

4. The method of claim 3, wherein the one or more changes comprise malformation or dysfunction of ocular drainage structures, one or more developmental anomalies, neural or non-neural cell degeneration, dysfunction, or death, or a combination thereof.

5. The method of claim 3, wherein the one or more changes result from environmental exposure, disease, aging, metabolic anomaly, mitochondrial anomaly, genetic mutation, or a combination thereof.

6. The method of claim 4, wherein the one or more developmental anomalies comprise changes to anterior chamber depth, pupil abnormalities, iridocorneal adhesions, trabecular meshwork, Schlemm's canal, cornea or a combination thereof.

7. The method of claim 4, wherein the cell degeneration or dysfunction comprises neurodegeneration and/or neural dysfunction in the subject's retinal ganglion cells.

8. The method of claim 4, wherein the ocular drainage structures comprise Schlemm's canal, trabecular meshwork, or a combination thereof.

9. The method of claim 1, wherein the ocular disease or disorder is a neurodegenerative disease or disorder.

10. The method of any claim 1, wherein the ocular disease or disorder is glaucoma or optic nerve disease.

11. The method of claim 1, wherein the condition is a neurodegenerative disease.

12. The method of claim 1, wherein the condition comprises late onset neurodegeneration.

13. The method of any claim 1, wherein the condition is Alzheimer's disease or Parkinson's disease.

14. The method of claim 1, wherein the disease or disorder linked to one or more Lmx1b mutations is a disease or disorder of the brain or kidney.

15. The method of claim 1, wherein the condition is age-related macular degeneration.

16. The method of claim 1, wherein the condition comprises a retinal pigment epithelium.

17. The method of claim 1, further comprising administering one or more additional therapeutic agents, or one or more pharmaceutical compositions thereof, to the subject.

18. The method of claim 17, wherein the one or more additional therapeutic agents are selected from the group consisting of an antioxidant agent, an anti-inflammatory agent, an agent that modulates metabolism, an agent that modulates the integrated stress response, an agent that modulates the unfolded protein response, an agent that modulates forms of autophagy, an agent that modulates the expression or activity of genes controlling or mediating antioxidant or other protective responses, a senolytic agent, an agent that modulates the mitochondria or mitophagy, an anti-aging agent, an agent that modulates intraocular pressure, a resilience-boosting agent, an antifibrotic agent, an agent that prevents epithelial mesenchymal transition or endothelial mesenchymal transition, a neuroprotective agent, a gene therapy agent, and a combination thereof.

19. The method of claim 18, wherein the agent that modulates metabolism reprograms or boosts metabolism.

20. The method of claim 18, wherein the antioxidant agent reduces oxidative stress and boosts antioxidant control.

21. The method of claim 18, wherein the gene therapy agent results in genome editing, genome reprogramming, epigenetic editing, epigenetic reprogramming, or a combination thereof.

22. The method of claim 18, wherein the agent that prevents epithelial mesenchymal transition or endothelial mesenchymal transition is an anti-transforming growth factor-β (TGFB) or ligand trap molecule.

23. The method of claim 18, where the one or more additional therapeutic agents are selected from the group consisting of nicotinamide, nicotinamide mononucleotide, nicotinamide adenine dinucleotide, nicotinamide ribose, pyrroloquinoline quinine, N-acetyl cysteine, and a combination thereof.

24. The method of claim 1, wherein the subject is a mammal.

25. A method of reducing or preventing elevated intraocular pressure, the method comprising administering a therapeutically effective amount of ethyl pyruvate, or a derivative or analog thereof, or a pharmaceutical composition thereof, to the subject.

26. The method of claim 25, further comprising administering one or more additional therapeutic agents, or one or more pharmaceutical compositions thereof, to the subject.

27. The method of claim 26, wherein the one or more additional therapeutic agents are selected from the group consisting of an antioxidant agent, an anti-inflammatory agent, an agent that modulates metabolism, a senolytic agent, an agent that modulates the mitochondria or mitophagy, an anti-aging agent, an agent that modulates intraocular pressure, a resilience-boosting agent, an antifibrotic agent, an agent that prevents epithelial mesenchymal transition or endothelial mesenchymal transition, a neuroprotective agent, a gene therapy agent, and a combination thereof.

28. A method of reducing or preventing damage or dysfunction to a subject's eye tissues, the method comprising administering a therapeutically effective amount of ethyl pyruvate, or a derivative or analog thereof, or a pharmaceutical composition thereof, to the subject.

29. The method of claim 28, further comprising administering one or more additional therapeutic agents, or one or more pharmaceutical compositions thereof, to the subject.

30. The method of claim 29, wherein the one or more additional therapeutic agents are selected from the group consisting of an antioxidant agent, an anti-inflammatory agent, an agent that modulates metabolism, a senolytic agent, an agent that modulates the mitochondria or mitophagy, an anti-aging agent, an agent that modulates intraocular pressure, a resilience-boosting agent, an antifibrotic agent, an agent that prevents epithelial mesenchymal transition or endothelial mesenchymal transition, a neuroprotective agent, a gene therapy agent, and a combination thereof.

31. A method of reducing or preventing damage or dysfunction to tissues involved in ocular fluid drainage in a subject, the method comprising administering a therapeutically effective amount of ethyl pyruvate, or a derivative or analog thereof, or a pharmaceutical composition thereof, to the subject.

32. The method of claim 31, further comprising administering one or more additional therapeutic agents, or one or more pharmaceutical compositions thereof, to the subject.

33. The method of claim 32, wherein the one or more additional therapeutic agents are selected from the group consisting of an antioxidant agent, an anti-inflammatory agent, an agent that modulates metabolism, a senolytic agent, an agent that modulates the mitochondria or mitophagy, an anti-aging agent, an agent that modulates intraocular pressure, a resilience-boosting agent, an antifibrotic agent, an agent that prevents epithelial mesenchymal transition or endothelial mesenchymal transition, a neuroprotective agent, a gene therapy agent, and a combination thereof.

34. A method of reducing or preventing developmental malformations or dysfunctions in a subject's eye, the method comprising administering a therapeutically effective amount of ethyl pyruvate, or a derivative or analog thereof, or a pharmaceutical composition thereof, to the subject.

35. The method of claim 34, further comprising administering one or more additional therapeutic agents, or one or more pharmaceutical compositions thereof, to the subject.

36. The method of claim 35, wherein the one or more additional therapeutic agents are selected from the group consisting of an antioxidant agent, an anti-inflammatory agent, an agent that modulates metabolism, a senolytic agent, an agent that modulates the mitochondria or mitophagy, an anti-aging agent, an agent that modulates intraocular pressure, a resilience-boosting agent, an antifibrotic agent, an agent that prevents epithelial mesenchymal transition or endothelial mesenchymal transition, a neuroprotective agent, a gene therapy agent, and a combination thereof.
